(19) United States
(12) Patent Application Publication
(10) Pub. No.: US 2017/0238987 A1
(43) Pub. Date: Aug. 24, 2017

(54) TISSUE TREATMENT WITH PLASMA ARC STREAM

(71) Applicant: Medident Technologies Inc., Surrey (CA)

(72) Inventor: Gilbert Fregoso, Fallbrook, CA (US)

(73) Assignee: Medident Technologies Inc., Surrey (CA)

(21) Appl. No.: 15/435,087

(22) Filed: Feb. 16, 2017

Related U.S. Application Data
(60) Provisional application No. 62/297,633, filed on Feb. 19, 2016.

(51) Int. Cl. A61B 18/04; A61B 18/16; A61B 18/12

(52) U.S. Cl. CPC A61B 18/042; A61B 18/1206; A61B 18/16; A61B 2018/1226

(57) ABSTRACT

A tissue treatment device includes a circuit driver, an electrode, and a plasma arc focusing element, all disposed within a housing. The circuit driver generates a plasma arc source signal. The electrode generates a plasma stream in response to the plasma arc source signal. The plasma arc focusing element focuses the plasma stream to pass through an outlet hole of the housing and onto a target spot on a target tissue of a treatment subject for heat treatment of the target tissue. In some embodiments, the circuit driver controls a temperature increase of the target tissue by modulating a fundamental frequency, power level, pulsing frequency, and/or pulsing duty cycle of the plasma arc source signal.

### RELATED APPLICATIONS

The application claims the benefit of U.S. Provisional Patent Application No. 62/297,633 filed on Feb. 19, 2016 and entitled “Tissue Treatment with Plasma Arc Stream”; which is hereby incorporated by reference for all purposes.

### BACKGROUND

Some types of medical conditions can be quite persistent and difficult or costly to treat. For example, treatment of a fungus on a person's toenail is typically performed in a doctor's office with an expensive laser device. Alternatively, an anti-fungal cream or ointment is applied to the infected area over a period of many months. A quick, low-cost, easy-to-use remedy treatment is needed.

### SUMMARY

In some embodiments, a tissue treatment device includes a housing, a circuit driver, an electrode, and a plasma arc focusing element. The housing includes an outlet hole. The circuit driver is disposed within the housing and generates a plasma arc source signal. The electrode is disposed within the housing, receives the plasma arc source signal, and generates a plasma stream in response to the plasma arc source signal. The plasma arc focusing element is disposed within the housing adjacent the electrode and the outlet hole. The plasma arc focusing element focuses the plasma stream to pass through the outlet hole and onto a target spot on a target tissue of a treatment subject for heat treatment of the target tissue. In some embodiments, the tissue treatment device further includes a plasma arc steering element disposed within the housing adjacent the electrode and the outlet hole. In some embodiments, the plasma arc steering element controls a direction of the plasma stream to scan the target spot over an area larger than the target spot. In some embodiments, the plasma arc focusing element and the plasma arc steering element are a single magnetic field coil. In some embodiments, the plasma arc focusing element is a first magnetic field coil, and the plasma arc steering element is a second magnetic field coil. In some embodiments, the plasma arc focusing element narrows the plasma stream to a diameter of 2 mm or less for the target spot. In some embodiments, the circuit driver controls a temperature increase of the target tissue that is caused by the plasma stream by modulating a fundamental frequency and power level of the plasma arc source signal. In some embodiments, the circuit driver further controls the temperature increase by modulating a pulsing frequency and pulsing duty cycle at which the plasma arc source signal is pulsed. In some embodiments, the plasma stream is capable of generating a cavitation pressure wave under a surface of the target tissue at the target spot. In some embodiments, the heat treatment is capable of treating a medical condition of the target tissue, such as a fungal infection, a bacterial infection, a pimple, or a fever blister. In some embodiments, the tissue treatment device further includes a grounding pad that is capable of contacting a skin surface of the treatment subject to form an electrical return path. In some embodiments, the tissue treatment device further includes a gas chamber disposed within the housing and that contains an inert gas that is flowed to the outlet hole, wherein the plasma stream is generated in the inert gas. In some embodiments, the tissue treatment device further includes a power storage source disposed within the housing and electrically connected to the circuit driver to provide electrical power to the circuit driver to generate the plasma arc source signal.

In some embodiments, a tissue treatment device includes a housing, a circuit driver, and an electrode. The housing includes an outlet hole. The circuit driver is disposed within the housing and generates a plasma arc source signal. The electrode is disposed within the housing, receives the plasma arc source signal, and generates a plasma stream in response to the plasma arc source signal. The plasma stream passes through the outlet hole and onto a target spot on a target tissue of a treatment subject for heat treatment of the target tissue. The circuit driver controls a temperature increase of the target tissue that is caused by the plasma stream by modulating a power level of the plasma arc source signal. In some embodiments, the circuit driver further controls the temperature increase by modulating a fundamental frequency of the plasma arc source signal. In some embodiments, the circuit driver further controls the temperature increase by modulating a pulsing frequency or pulsing duty cycle at which the plasma arc source signal is pulsed. In some embodiments, the tissue treatment device further includes a plasma arc focusing element disposed within the housing adjacent the electrode and the outlet hole and that focuses the plasma stream to pass through the outlet hole and onto the target spot.

Some embodiments are characterized by a method in which a circuit driver (disposed within a housing of a tissue treatment device) generates a plasma arc source signal. An electrode (disposed within the housing) generates a plasma stream in response to the plasma arc source signal. A plasma arc focusing element (disposed within the housing) generates a magnetic field source signal. The magnetic field source signal focuses and directs the plasma stream through an outlet hole of the housing and onto a target spot of a predetermined size on a target tissue of a treatment subject for heat treatment of the target tissue. In some embodiments, the directing of the plasma stream is altered to scan the target spot over an area larger than the target spot. In some embodiments, a temperature increase of the target tissue (that is caused by the plasma stream) is controlled by the circuit driver modulating a fundamental frequency and power level of the plasma arc source signal. In some embodiments, the temperature increase is controlled by also modulating a pulsing frequency and pulsing duty cycle at which the plasma arc source signal is pulsed.

### BRIEF DESCRIPTION OF THE DRAWINGS

FIG. 1 is a simplified plan drawing of an example treatment device for treating living tissue with a plasma arc stream in accordance with an embodiment of the present invention.

FIG. 2 is a simplified schematic block diagram of the example treatment device shown in FIG. 1 in accordance with an embodiment of the present invention.

FIG. 3 is a simplified schematic block diagram of the example treatment device shown in FIG. 1 in accordance with another embodiment of the present invention.

FIG. 4 is a simplified plan drawing of another example treatment device for treating living tissue with a plasma arc stream in accordance with an embodiment of the present invention.

FIG. 5 is a simplified schematic block diagram of the example treatment device shown in FIG. 4 in accordance with an embodiment of the present invention.

FIG. 6 is a simplified schematic block diagram of the example treatment device shown in FIG. 4 in accordance with another embodiment of the present invention.

FIG. 7 is a simplified plan drawing of another example treatment device for treating living tissue with a plasma arc stream in accordance with an embodiment of the present invention.

FIG. 8 is a simplified schematic block diagram of the example treatment device shown in FIG. 7 in accordance with an embodiment of the present invention.

FIG. 9 is a simplified schematic block diagram of the example treatment device shown in FIG. 7 in accordance with another embodiment of the present invention.

### DETAILED DESCRIPTION

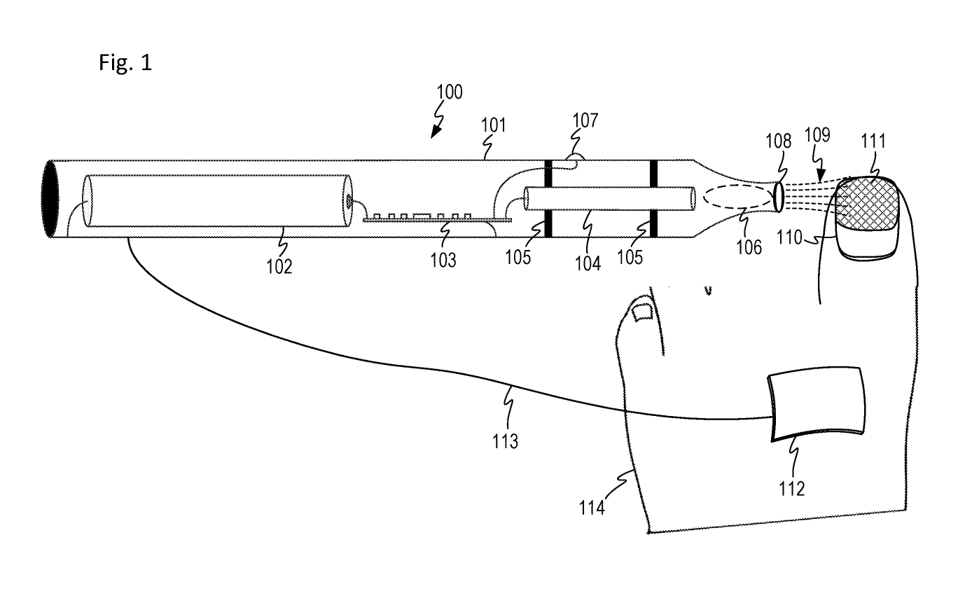

FIGS. 1-9 illustrate example embodiments of treatment devices for treating living tissue with a plasma arc stream. Each example treatment device enables a quick, low-cost toenail fungus treatment that is amenable to being used by a consumer as a simple home or doctor office remedy.

Each example treatment device is described as being used to treat a fungal infection on the surface of a human's toenail. However, some embodiments of the treatment devices may also be used to treat any appropriate tissue for any appropriate condition. Some examples of appropriate conditions that can be treated are fungal infections, bacterial infections, pimples, toenail fungus, onychomycosis, athlete's foot, and fever blisters, among others.

FIGS. 1, 2 and 3 show an example treatment device 100 for treating a medical condition (e.g., killing a fungal infection) at a pinpoint target area of a living tissue (e.g., a human foot, toe or toenail) with a plasma arc stream. In the illustrated embodiment of FIG. 1, the treatment device 100 generally includes an elongated housing 101, a power source (e.g., a battery) 102, a circuit driver (or circuit driver board) 103, one or more electrode 104, one or more insulator 105, a plasma chamber 106, a start/stop switch 107, and a grounding pad 112. Additional components may also be included, but are not shown for simplicity.

In some embodiments, the housing 101 has a generally tubular shape with an appropriate diameter throughout most of its length to contain most of the other components, but tapers to a smaller diameter near an outlet hole 108 at one end. The general shape of the housing 101 is appropriate for being held in a person's hand. The housing 101 is generally made of any appropriate material, such as aluminum, other metal, plastic, etc. In some embodiments, the housing 101 serves as an electrical ground return path between the plasma chamber 106, the circuit driver board 103, and the battery 102. The housing 101 contains, and provides a mounting support for, the battery 102, the circuit driver board 103, the insulators 105, the plasma chamber 106, the start/stop switch 107, and the electrode 104. In some embodiments, the plasma chamber 106 is a defined volume (i.e., a plasma region) wherein the plasma is generated within the interior of the housing 101 proximate to the outlet hole 108.

The grounding pad 112 is electrically connected to the housing 101 or the circuit driver board 103 (through the housing 101) via an electrical conductor 113, e.g., a wire. In some embodiments, the grounding pad 112 contacts a skin surface of the treatment subject (e.g., a person, animal, etc. having the target tissue to be treated) as a ground return from the treatment subject through the electrical conductor 113 to the housing 101 or the circuit driver board 103. The ground return helps the plasma arc to be directed to the target tissue of the treatment subject. In some embodiments, the grounding pad 112 is attached to the treatment subject, e.g., with a conductive adhesive to an area of the treatment subject's skin near the target tissue. For example, if the target tissue is a person's toenail, then the grounding pad 112 can be attached to the person's foot 114 relatively close to the target toenail as shown.

In some embodiments, the circuit driver board 103 is made of solid state integrated semiconductor passive and active components mounted in an IC (integrated circuit) chip and/or on a PCB (printed circuit board). The circuit driver board 103 is electrically connected to the battery 102, the electrode 104, and the housing 101. With power supplied by the battery 102, the circuit driver board 103 creates a high voltage, low current plasma arc source signal that is applied to the electrode 104. In some embodiments, the power delivered to the electrode 104 is from 0.1 W to 10 W, or from 4 W to 7 W, or from 3 W to 5 W.

In some embodiments, the electrode 104 is a plasma arc center electrode. The electrode 104 is made of any appropriate material, such as stainless steel, platinum, gold, silver, other metal, conductive ceramic, a mixed combination of metals, etc. The electrode 104 is electrically connected to the circuit driver board 103. Additionally, one end of the electrode 104 is positioned in or adjacent to the plasma chamber 106. The high voltage, low current plasma arc source signal received from the circuit driver board 103 causes the electrode 104 to strike or generate a plasma in a gas (e.g., oxygen, helium, argon, nitrogen, etc.) in the plasma chamber 106. In some embodiments, the plasma arc stream comprises reactive species, which are formed from the ionization and reaction of the gas. The reactive species can be tuned by changing the composition of the gas and/or adjusting the parameters (e.g., voltage, current, power, fundamental frequency, pulsing frequency, pulsing duty cycle, pulse shape, etc.) of the plasma arc source signal provided to the electrode 104. In some embodiments, the plasma chamber 106 is composed of materials that are resistant to reaction from the plasma. Some examples of materials that the plasma chamber 106 can be made of include aluminum, stainless steel, conductive glass, conductive plastic, etc.

In some embodiments, the insulators 105 are made of insulating plastic materials, ceramics, glass or other appropriate insulating materials. The insulators 105 contact the inside surface of the housing 101 and the outer surface of the electrode 104 (near the two ends of the electrode 104) to support the electrode 104 in a spaced apart, generally concentric, relationship with the housing 101. The insulator 105, thus, electrically and thermally insulates the electrode 104 from the inside surface of the housing 101.

In some embodiments, the battery 102 is any appropriate rechargeable or non-rechargeable power storage source (e.g., a Li-ion battery, an alkaline battery, etc.) capable of producing an appropriate voltage (e.g., 0.8 volts dc to 7.4 volts dc). The battery 102 is electrically connected to the circuit driver board 103 to provide electrical power to the circuit driver board 103.

In some embodiments, the start/stop switch (or button) 107 is mounted through the housing 101, so that it is exposed and accessible on the outside of the housing 101. Inside the housing 101, the start/stop switch 107 is electrically connected to the circuit driver board 103. When turned on or off (e.g., by being pressed or switched by a user), the start/stop switch 107 activates or deactivates, respectively, the operation of the circuit driver board 103.

When activated, the circuit driver board 103 provides the plasma arc source signal to the electrode 104. In response, the electrode 104 generates the plasma in the gas in the plasma chamber 106. As the gas flows through the plasma chamber 106, the generated plasma passes out through the outlet hole 108 as a plasma arc stream 109. When the outlet hole 108 is held near the target tissue 110 (e.g., a person's toenail) the plasma arc stream 109 heat treats the target tissue (e.g., to kill a fungus 111 on the target tissue 110). In some embodiments, the “target temperature” of the target tissue 110 being treated rises during treatment to greater than 100° F., or greater than 110° F., or greater than 120° F., or greater than 130° F., or greater than 140° F., or greater than 150° F., or greater than 160° F., or from 100° F. to 180° F., or from 120° F. to 180° F., or from 120° F. to 160° F., or from 130° F. to 150° F. In some embodiments, the temperature differential for the temperature rise of the target tissue 110 is between 30° F. and 100° F.

In some embodiments, the heat generated in the target tissue 110 due to the interaction with the plasma arc stream causes a change of pressure in the tissue being treated, which can lead to cavitation that destroys surrounding tissue. Cavitation, as used herein, is the formation of vapor cavities in a liquid that are the consequence of forces acting upon the liquid. Cavitation generally occurs when a liquid is subjected to rapid changes of pressure that cause the formation of cavities where the pressure is relatively low. When subjected to higher pressure from without, the voids implode and can generate an intense shock wave, or cavitation pressure wave, under the surface of the target tissue at the target spot. Within the context of the present disclosure, the shock wave can damage or destroy the tissue surrounding the target tissue 110 interacting the with plasma.

In some embodiments, the plasma arc source signal provided by the circuit driver board 103 to the electrode 104 is continuous wave or pulsed. In some embodiments, the plasma arc source signal provided to the electrode 104 that is generating the plasma has a time-averaged power from 0.1 W to 10 W, or from 4 W to 7 W. In some embodiments, the plasma arc source signal provided to the electrode 104 that is generating the plasma has a fundamental frequency from 10 kHz to 1 MHz, or from 50 kHz to 500 kHz, or is approximately 100 kHz. In some embodiments, the plasma arc source signal provided to electrode 104 that is generating the plasma is pulsed with a repetitive, modulation or pulsing frequency from 5 Hz to 100 Hz, or 15 Hz to 500 Hz. In some embodiments, the plasma arc source signal provided to electrode 104 that is generating the plasma is pulsed and has a pulsing duty cycle (i.e., the fraction of time the instantaneous power of the plasma arc source signal is on divided by the fraction of time the instantaneous power of the plasma arc source signal is off) from 1% to 10%, or from 10% to 90%, or from 20% to 80%, or from 30% to 70%, or from 40% to 60%, or from 10% to 50%, or from 50% to 90%, or from 10% to 30%, or from 70% to 90%. In some embodiments, the plasma arc source signal provided to the electrode 104 is pulsed with a pulse duration of 50 ns to 200 microseconds, or 10 ns to 300 microseconds. In some embodiments, the shape of the pulses in a frequency modulated plasma arc source signal provided to the electrode 104 that is generating the plasma is a square pulse, a rectangular pulse, a saw tooth pulse, a triangle pulse, or other shape. Not to be limited by theory, the combination of the time-averaged power, the fundamental frequency, the pulsing frequency, the pulsing duty cycle, and the pulse shape generally determine the average temperature rise of the target tissue interacting with the plasma arc stream. In some embodiments, the time-averaged power, the fundamental frequency, the pulsing frequency, the pulsing duty cycle, and the pulse shape of the plasma arc source signal provided to the electrode 104 are chosen or tuned to generate a particular predetermined average temperature rise in the target tissue that is interacting with the resulting plasma arc stream. In this manner, the circuit driver board 103 controls the temperature or temperature increase of the target tissue by controlling, adjusting or modulating these parameters. In some embodiments, the target temperature can be different. For example, the temperatures typically needed to destroy fungal infections are different from the temperatures typically needed to destroy bacterial infections. In some embodiments, different plasma parameters (e.g., time-average power, pulsing frequency, etc.) are needed to generate a particular predetermined target temperature in a target tissue interacting with the plasma arc stream. For example, a fungal infection under a toenail would require more time-averaged power than the tissue of a pimple, to generate a 140° F. target temperature.

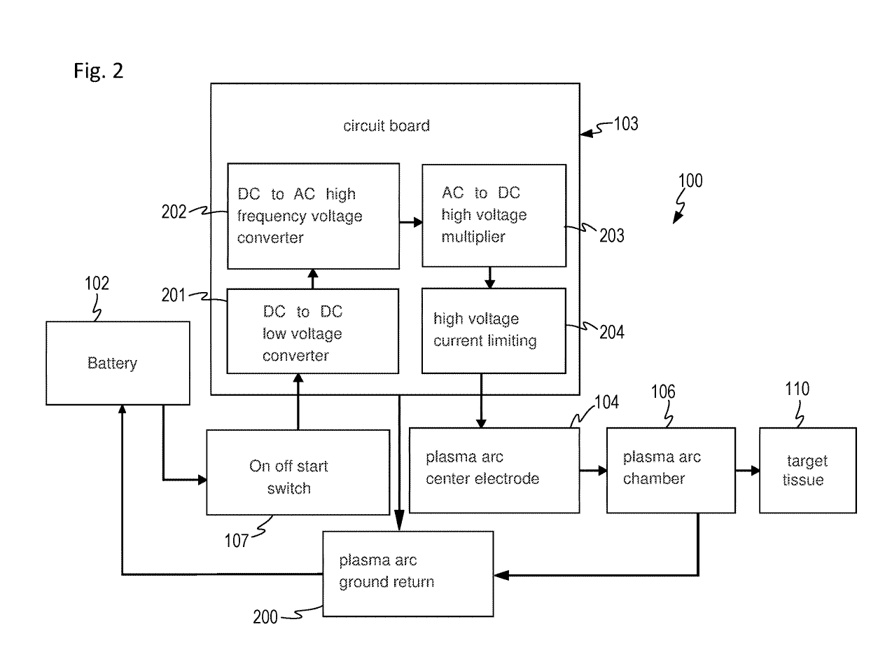

FIG. 2 shows an embodiment of the treatment device 100 having the battery 102, the circuit driver board 103, the one or more electrode 104, the plasma (arc) chamber 106, and the start/stop switch 107. In some embodiments, these components are generally the same or similar to the components (having the same reference numbers) described above with reference to FIG. 1. Additionally, in some embodiments, the treatment device 100 also has a plasma arc ground return 200. Furthermore, in some embodiments, the circuit driver board 103 generally has a DC to DC low voltage converter 201, a DC to AC high frequency voltage converter 202, an AC to DC high voltage multiplier 203, and a high voltage current limiting element 204.

In some embodiments, the plasma arc ground return 200 is a wire or other conductor that extends between and electrically connects the plasma arc chamber 106, the circuit driver board 103, and the battery 102 to complete the electrical circuit. In some embodiments, the housing 101 serves as the plasma arc ground return 200. In some embodiments, another ground return (not shown in FIG. 2) is provided for the grounding pad 112 attached to the treatment subject, as described above, to assist in ensuring that the plasma arc stream reaches the target tissue.

In some embodiments, the DC to DC low voltage converter 201 receives a voltage from the battery 102 and converts it to a DC voltage(s) that is utilized by the components of the circuit driver board 103.

In some embodiments, the DC to AC high frequency voltage converter 202 is a relatively efficient means in a relatively small package size for receiving the DC voltage from the DC to DC low voltage converter 201 and generating an AC high frequency voltage signal that is needed for plasma arc signal generation.

In some embodiments, the AC to DC high voltage multiplier 203 receives the AC high frequency voltage signal and converts it to further increase the voltage and rectify it to a DC voltage.

In some embodiments, the high voltage current limiting element 204 receives the DC voltage and limits the power of the plasma arc, so as to protect the target tissue 110 from temperatures greater than the target temperature.

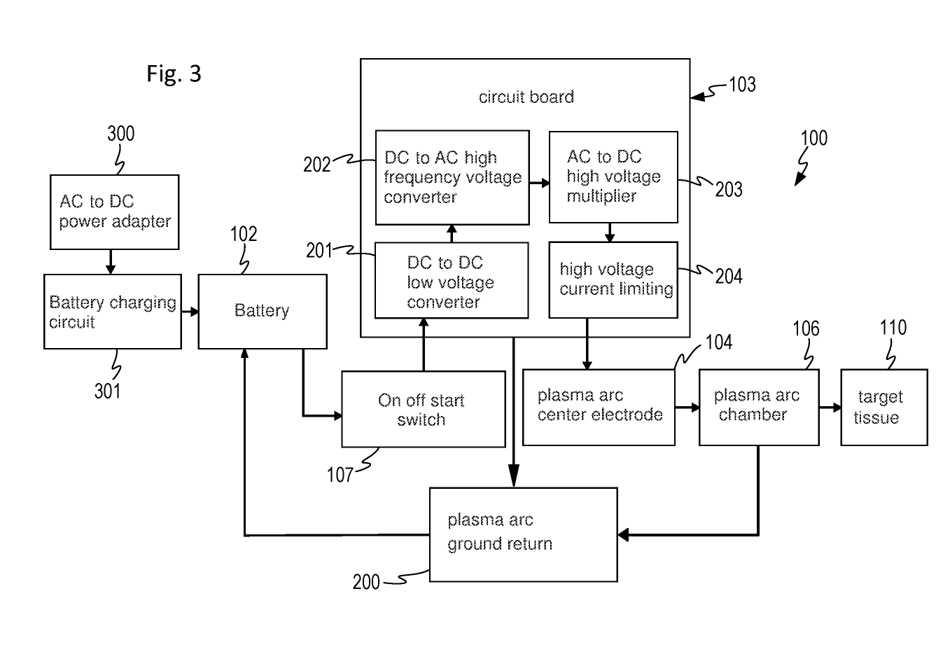

FIG. 3 shows an embodiment of the treatment device 100 having the battery 102, the circuit driver board 103, the one or more electrode 104, the plasma (arc) chamber 106, the start/stop switch 107, the plasma arc ground return 200, the DC to DC low voltage converter 201, the DC to AC high frequency voltage converter 202, the AC to DC high voltage multiplier 203, and the high voltage current limiting element 204. In some embodiments, these components are generally the same or similar to the components (having the same reference numbers) described above with reference to FIGS. 1 and 2. In some embodiments, the treatment device 100 also has or is connected to an AC to DC power adapter 300 and a battery charging circuit 301.

In some embodiments, the AC to DC power adapter 300 is used for charging the battery 102 and for powering the treatment device 100 when plugged into a AC outlet in the event the battery 102 was totally or partially discharged. In some embodiments, the AC to DC power adapter 300 is external or internal to the housing 101.

In some embodiments, the battery charging circuit 301 is incorporated onto the circuit driver board 103 for quick charging of the battery 102. In some embodiments, the battery charging circuit 301 is external or internal to the housing 101.

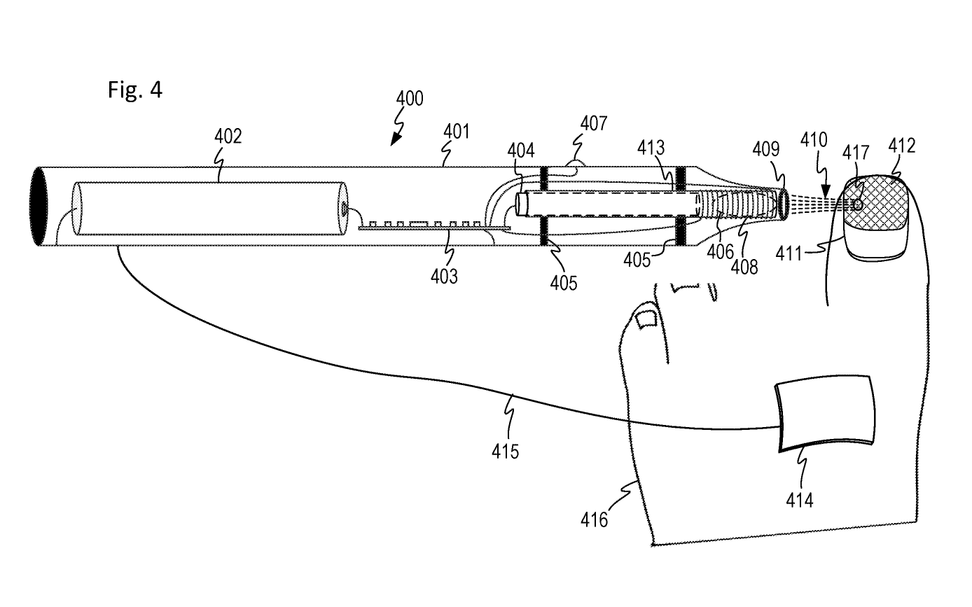

FIGS. 4, 5 and 6 show another example treatment device 400 for treating a medical condition (e.g., killing a fungal infection) at a pinpoint target area of a living tissue (e.g., a human foot, toe or toenail) with a plasma arc stream. In the illustrated embodiment of FIG. 4, the treatment device 400 generally includes an elongated housing 401, a power source (e.g., a battery) 402, a circuit driver (or circuit driver board) 403, one or more electrode 404, one or more insulator 405, a plasma chamber 406, a start/stop switch 407, and one or more magnetic field (steering or focusing) coil 408, and a grounding pad 414. Additional components may also be included, but are not shown for simplicity.

In some embodiments, the housing 401 has a generally tubular shape with an appropriate diameter throughout most of its length to contain most of the other components, but tapers to a smaller diameter near an outlet hole 409 at one end. The general shape of the housing 401 is appropriate for being held in a person's hand. The housing 401 is generally made of any appropriate material, such as aluminum, other metal, plastic, etc. In some embodiments, the housing 401 serves as an electrical ground return path between the plasma chamber 406, the circuit driver board 403, and the battery 402. The housing 401 contains, and provides a mounting support for, the battery 402, the circuit driver board 403, the insulators 405, the plasma chamber 406, the start/stop switch 407, the electrode 404, and the magnetic field coil 408. In some embodiments, the plasma chamber 406 is a defined volume (i.e., a plasma region) wherein the plasma is generated within the interior of the housing 401 proximate to the outlet hole 409.

The grounding pad 414 is electrically connected to the housing 401 or the circuit driver board 403 (through the housing 401) via an electrical conductor 415, e.g., a wire. In some embodiments, the grounding pad 414 contacts a skin surface of the treatment subject (e.g., a person, animal, etc. having the target tissue to be treated) as a ground return from the treatment subject through the electrical conductor 415 to the housing 401 or the circuit driver board 403. The ground return helps the plasma arc to be directed to the target tissue of the treatment subject. In some embodiments, the grounding pad 414 is attached to the treatment subject, e.g., with a conductive adhesive to an area of the treatment subject's skin near the target tissue. For example, if the target tissue is a person's toenail, then the grounding pad 414 can be attached to the person's foot 416 relatively close to the target toenail as shown.

In some embodiments, the circuit driver board 403 is made of solid state integrated semiconductor passive and active components mounted in an IC (integrated circuit) chip and/or on a PCB (printed circuit board). The circuit driver board 403 is electrically connected to the battery 402, the electrode 404, the magnetic field coil 408, and the housing 401. With power supplied by the battery 402, the circuit driver board 403 creates a high voltage, low current plasma arc source signal that is applied to the electrode 404 and a magnetic field source signal that is applied to the magnetic field coil 408. The power delivered to the electrode 404 is the same or similar to that described above for the embodiments of FIGS. 1-3. In some embodiments, the current delivered to the magnetic field coil 408 is from 50 mA to 500 mA or greater.

In some embodiments, the electrode 404 is a plasma arc center electrode. The electrode 404 is made of any appropriate material, such as stainless steel, platinum, gold, silver, other metal, conductive ceramic, a mixed combination of metals, etc. The electrode 404 is electrically connected to the circuit driver board 403. Additionally, one end of the electrode 404 is positioned in or adjacent to the plasma chamber 406. The plasma arc source signal received from the circuit driver board 403 causes the electrode 404 to strike or generate a plasma in a gas (e.g., oxygen, helium, argon, nitrogen, etc.) in the plasma chamber 406. In some embodiments, the plasma arc stream comprises reactive species, which are formed from the ionization and reaction of the gas. The reactive species can be tuned by changing the composition of the gas and/or adjusting the parameters (e.g., voltage, current, power, fundamental frequency, pulsing frequency, pulsing duty cycle, pulse shape, etc.) of the plasma arc source signal provided to the electrode 404 and the magnetic field source signal applied to the magnetic field coil 408. In some embodiments, the plasma chamber 406 is composed of materials that are resistant to reaction from the plasma, e.g., as described above for the embodiments of FIGS. 1-3.

In some embodiments, the magnetic field coil 408 is any appropriate plasma arc steering and/or focusing element, such as a multi turn copper wire that is wrapped around a bobbin (e.g., a sleeve 413). The magnetic field coil 408 is placed close or adjacent to the electrode 404, the plasma chamber (region) 406, the outlet hole 409, and/or the plasma arc source signal. In some embodiments, the magnetic field coil 408 generally surrounds a substantial portion of the plasma chamber 406 adjacent the outlet hole 409. The magnetic field source signal received from the circuit driver board 403 causes the magnetic field coil 408 to control the direction of the plasma arc source signal to direct the plasma field from the plasma chamber 406 onto a target spot 417 on the target tissue 411.

In some embodiments, the magnetic field coil 408 represents at least two separate plasma arc steering and focusing elements. In some embodiments, there is a steering coil and a focusing coil, each comprising a magnetic field coil.

Not to be limited by theory, the spot size will be a primary factor in determining the power density of the plasma arc stream where it interacts with the target tissue being treated. In some embodiments, the spot size can be used to generate a particular average temperature rise in the tissue that is interacting with the plasma arc stream within the target spot 417. With a smaller target spot size, the treatment subject is typically able to withstand a greater temperature and associated pain. In some embodiments, the target spot diameter has a predetermined size from 0.1 mm to 10 mm, from 0.5 to 5 mm, or from 1 mm to 2 mm, or 10, 5 or 2 mm or less.

In some embodiments, the device is scanned manually by the user (i.e., moving the target spot 417 manually to interact with all of the areas of the target tissue 411 intended to be treated). In some embodiments, the plasma arc stream rasters back and forth over the tissue to be treated, e.g., using the steering coil to alter or control the direction of the plasma arc stream and scan the target spot 417 over an area larger than the target spot 417. Not to be limited by theory, the temperature of the tissue interacting with the plasma arc stream can be impacted or controlled by the amount of time that the plasma arc stream is interacting with the target tissue 411, up until an amount of time where a steady state temperature is reached. In some embodiments, the scanning rate of the plasma arc stream over the target tissue 411 being treated is chosen to generate a particular average temperature rise in the target tissue 411 that is interacting with the plasma arc stream.

In some embodiments, the insulators 405 are made of insulating plastic materials, ceramics, glass or other appropriate insulating materials. The insulators 405 contact the inside surface of the housing 401 and the outer surface of the sleeve 413 or the electrode 404 (near the two ends of the electrode 404) to support the electrode 404 (and the magnetic field coil 408) in a spaced apart, generally concentric, relationship with the housing 401. The insulators 405, thus, electrically and thermally insulate the electrode 404 (and the magnetic field coil 408) from the inside surface of the housing 401.

In some embodiments, the battery 402 is any appropriate rechargeable or non-rechargeable power storage source (e.g., a Li-ion battery, an alkaline battery, etc.) capable of producing an appropriate voltage (e.g., 0.8 volts dc to 7.4 volts dc). The battery 402 is electrically connected to the circuit driver board 403 to provide electrical power to the circuit driver board 403.

In some embodiments, the start/stop switch (or button) 407 is mounted through the housing 401, so that it is exposed and accessible on the outside of the housing 401. Inside the housing 401, the start/stop switch 407 is electrically connected to the circuit driver board 403. When turned on or off (e.g., by being pressed or switched by a user), the start/stop switch 407 activates or deactivates, respectively, the operation of the circuit driver board 403.

When activated, the circuit driver board 403 provides the plasma arc source signal to the electrode 404 and the magnetic field source signal to the magnetic field coil 408. In response, the electrode 404 and the magnetic field coil 408 generate the plasma in the gas in the plasma chamber 406. As the gas flows through the plasma chamber 406, the generated plasma passes out through the outlet hole 409 as a plasma arc stream 410. When the outlet hole 409 is held near the target tissue 411 (e.g., a person's toenail) the plasma arc stream 410 heat treats the target tissue (e.g., to kill a fungus 412 on the target tissue 411). In some embodiments, the target temperature and the temperature differential are the same or similar to that described above for embodiments of FIGS. 1-3. In some embodiments, the heat generated in the target tissue 411 causes the cavitation, and resulting effects, as described above.

In some embodiments, the plasma arc source signal provided by the circuit driver board 403 to the electrode 404 is continuous wave or pulsed. In some embodiments, the plasma arc source signal provided to the electrode 404 has a time-averaged power, a fundamental frequency, a pulsing frequency, a pulsing duty cycle, pulse duration, and/or a pulse shape that are the same or similar to the parameters described above for embodiments of FIGS. 1-3. Not to be limited by theory, the combination of the time-averaged power, the fundamental frequency, the pulsing frequency, the pulsing duty cycle, and the pulse shape generally determine the average temperature rise of the target tissue interacting with the plasma arc stream. In some embodiments, the time-averaged power, the fundamental frequency, the pulsing frequency, the pulsing duty cycle, and the pulse shape of the plasma arc source signal provided to the electrode 404 are chosen or tuned to generate a particular predetermined average temperature rise in the target tissue that is interacting with the resulting plasma arc stream. In this manner, the circuit driver board 403 controls the temperature or temperature increase of the target tissue by controlling, adjusting or modulating these parameters. In some embodiments, the target temperature can be different, as described above. In some embodiments, different plasma parameters (e.g., time-average power, pulsing frequency, etc.) are needed to generate a particular predetermined target temperature in a target tissue interacting with the plasma arc stream.

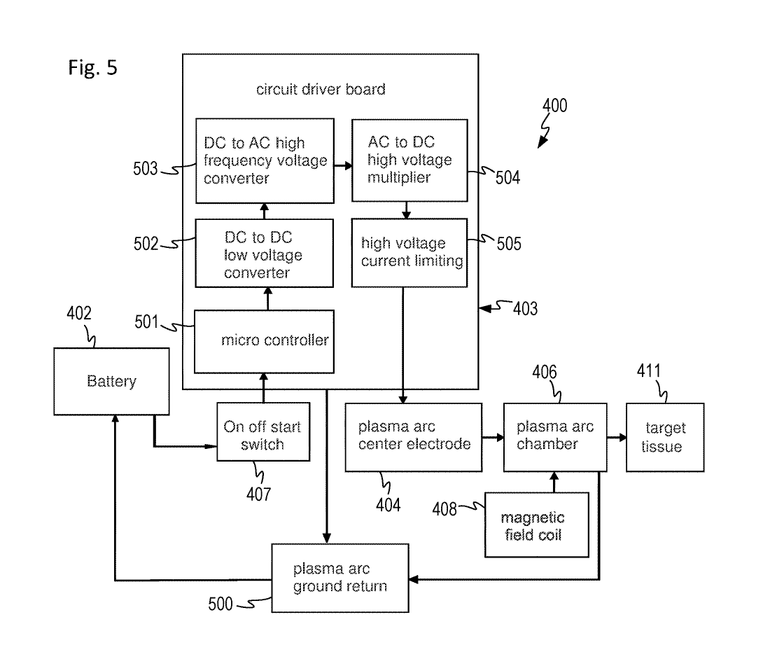

FIG. 5 shows an embodiment of the treatment device 400 having the battery 402, the circuit driver board 403, the one or more electrode 404, the plasma (arc) chamber 406, the start/stop switch 407, and the magnetic field coil 408. In some embodiments, these components are generally the same or similar to the components (having the same reference numbers) described above with reference to FIG. 4. Additionally, in some embodiments, the treatment device 400 also has a plasma arc ground return 500. Furthermore, in some embodiments, the circuit driver board 403 generally has a microcontroller 501, a DC to DC low voltage converter 502, a DC to AC high frequency voltage converter 503, an AC to DC high voltage multiplier 504, and a high voltage current limiting element 505.

In some embodiments, the plasma arc ground return 500 is a wire or other conductor that extends between and electrically connects the plasma arc chamber 406, the circuit driver board 403, and the battery 402 to complete the electrical circuit. In some embodiments, the housing 401 serves as the plasma arc ground return 500. In some embodiments, another ground return (not shown in FIG. 5) is provided for the grounding pad 414 attached to the treatment subject, as described above, to assist in ensuring that the plasma arc stream reaches the target tissue.

In some embodiments, the microcontroller 501 controls all functions of operation of the treatment device 400, including the magnetic field coil 408 and the direction of the plasma arc signal. Under this control, the microcontroller 501 can narrow the plasma field to a pinpoint or open it to a wider field.

In some embodiments, the DC to DC low voltage converter 502 receives a voltage from the battery 402 and converts it to a DC voltage(s) that is utilized by the components of the circuit driver board 403.

In some embodiments, the DC to AC high frequency voltage converter 503 is a relatively efficient means in a relatively small package size for receiving the DC voltage from the DC to DC low voltage converter 502 and generating an AC high frequency voltage signal that is needed for plasma arc signal generation.

In some embodiments, the AC to DC high voltage multiplier 504 receives the AC high frequency voltage signal and converts it to further increase the voltage and rectify it to a DC voltage.

In some embodiments, the high voltage current limiting element 505 receives the DC voltage and limits the power of the plasma arc, so as to protect the target tissue 411 from temperatures greater than the target temperature.

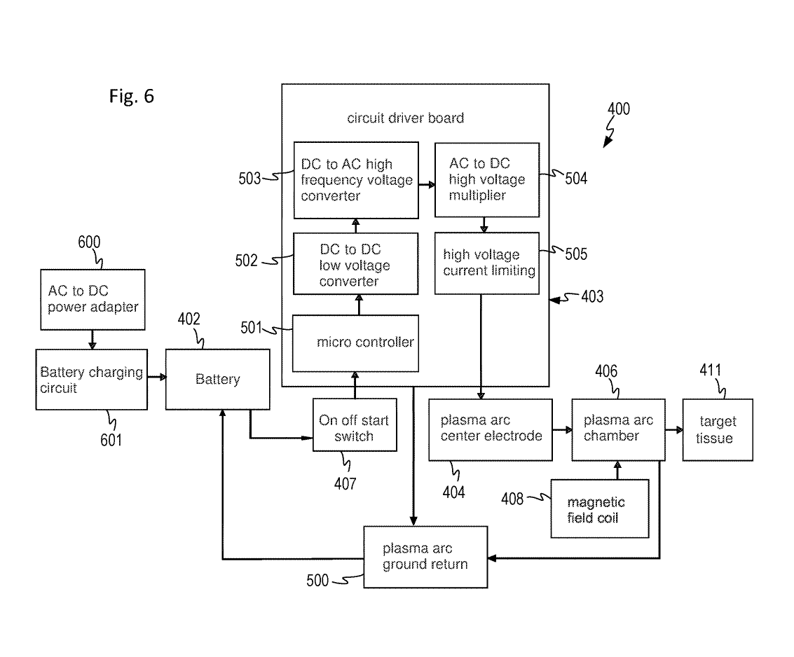

FIG. 6 shows an embodiment of the treatment device 400 having the battery 402, the circuit driver board 403, the one or more electrode 404, the plasma (arc) chamber 406, the start/stop switch 407, the magnetic field coil 408, the plasma arc ground return 500, the microcontroller 501, the DC to DC low voltage converter 502, the DC to AC high frequency voltage converter 503, the AC to DC high voltage multiplier 504, and the high voltage current limiting element 505. In some embodiments, these components are generally the same or similar to the components (having the same reference numbers) described above with reference to FIGS. 4 and 5. In some embodiments, the treatment device 400 also has or is connected to an AC to DC power adapter 600 and a battery charging circuit 601.

In some embodiments, the AC to DC power adapter 600 is used for charging the battery 402 and for powering the treatment device 400 when plugged into a AC outlet in the event the battery 402 was totally or partially discharged. In some embodiments, the AC to DC power adapter 600 is external or internal to the housing 401.

In some embodiments, the battery charging circuit 601 is incorporated onto the circuit driver board 403 for quick charging of the battery 402. In some embodiments, the battery charging circuit 601 is external or internal to the housing 401.

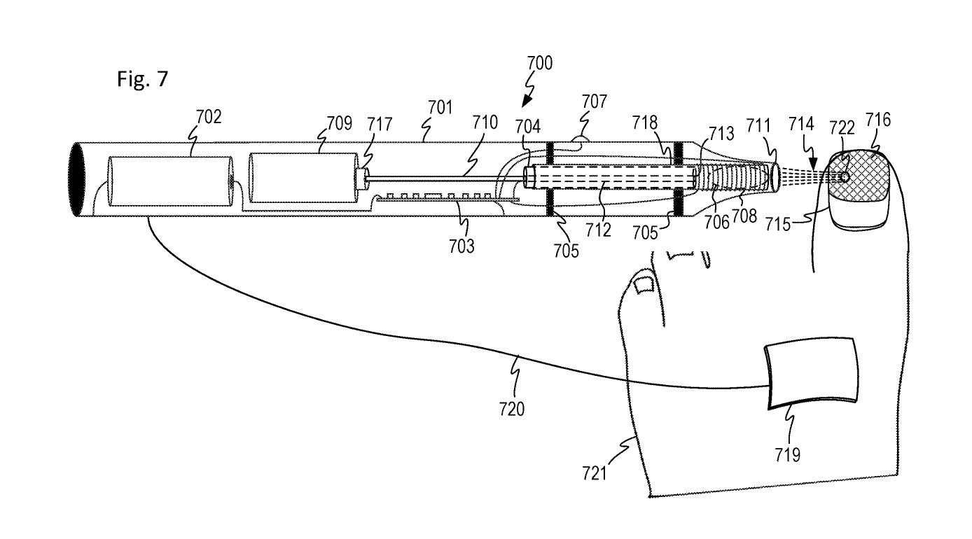

FIGS. 7, 8 and 9 show another example treatment device 700 for treating a medical condition (e.g., killing a fungal infection) at a pinpoint target area of a living tissue (e.g., a human foot, toe or toenail) with a plasma arc stream. In the illustrated embodiment of FIG. 7, the treatment device 700 generally includes an elongated housing 701, a power source (e.g., a battery) 702, a circuit driver (or circuit driver board) 703, one or more electrode 704, one or more insulator 705, a plasma chamber 706, a start/stop switch 707, one or more magnetic field (steering or focusing) coil 708, an inert gas chamber 709, a gas tube 710, and a grounding pad 719. Additional components may also be included, but are not shown for simplicity.

In some embodiments, the housing 701 has a generally tubular shape with an appropriate diameter throughout most of its length to contain most of the other components, but tapers to a smaller diameter near an outlet hole 711 at one end. The general shape of the housing 701 is appropriate for being held in a person's hand. The housing 701 is generally made of any appropriate material, such as aluminum, other metal, plastic, etc. In some embodiments, the housing 701 serves as an electrical ground return path between the plasma chamber 706, the circuit driver board 703, and the battery 702. The housing 701 contains, and provides a mounting support for, the battery 702, the circuit driver board 703, the insulators 705, the plasma chamber 706, the start/stop switch 707, the electrode 704, the magnetic field coil 708, the inert gas chamber 709, and the gas tube 710. In some embodiments, the plasma chamber 706 is a defined volume (i.e., a plasma region) wherein the plasma is generated within the interior of the housing 701 proximate to the outlet hole 711.

The grounding pad 719 is electrically connected to the housing 701 or the circuit driver board 703 (through the housing 701) via an electrical conductor 720, e.g., a wire. In some embodiments, the grounding pad 719 contacts a skin surface of the treatment subject (e.g., a person, animal, etc. having the target tissue to be treated) as a ground return from the treatment subject through the electrical conductor 720 to the housing 701 or the circuit driver board 703. The ground return helps the plasma arc to be directed to the target tissue of the treatment subject. In some embodiments, the grounding pad 719 is attached to the treatment subject, e.g., with a conductive adhesive to an area of the treatment subject's skin near the target tissue. For example, if the target tissue is a person's toenail, then the grounding pad 719 can be attached to the person's foot 721 relatively close to the target toenail as shown.

In some embodiments, the circuit driver board 703 is made of solid state integrated semiconductor passive and active components mounted in an IC (integrated circuit) chip and/or on a PCB (printed circuit board). The circuit driver board 703 is electrically connected to the battery 702, the electrode 704, the magnetic field coil 708, a magnetic solenoid gas flow valve 717, and the housing 701. With power supplied by the battery 702, the circuit driver board 703 creates a high voltage, low current plasma arc source signal that is applied to the electrode 704, a magnetic field source signal that is applied to the magnetic field coil 708, and a valve control signal that is applied to the gas flow valve 717. The voltage, current and/or power delivered to the electrode 704 and/or the magnetic field coil 708 is the same or similar to that described above for the embodiments of FIGS. 1-3 and 4-6.

In some embodiments, the electrode 704 is a plasma arc center electrode. The electrode 704 is made of any appropriate material, such as stainless steel, platinum, gold, silver, other metal, conductive ceramic, a mixed combination of metals, etc. The electrode 704 is electrically connected to the circuit driver board 703. Additionally, one end of the electrode 704 is positioned in or adjacent to the plasma chamber 706. The plasma arc source signal received from the circuit driver board 703 causes the electrode 704 to strike or generate a plasma in a gas (e.g., oxygen, helium, argon, nitrogen, etc.) in the plasma chamber 706. In some embodiments, the plasma arc stream comprises reactive species, which are formed from the ionization and reaction of the gas. The reactive species can be tuned by changing the composition of the gas and/or adjusting the parameters (e.g., voltage, current, power, fundamental frequency, pulsing frequency, pulsing duty cycle, pulse shape, etc.) of the plasma arc source signal provided to the electrode 704 and the magnetic field source signal applied to the magnetic field coil 708. In some embodiments, the plasma chamber 706 is composed of materials that are resistant to reaction from the plasma, e.g., as described above for the embodiments of FIGS. 1-3 and 4-6.

In some embodiments, the magnetic field coil 708 is any appropriate plasma arc steering and/or focusing element, such as a multi turn copper wire that is wrapped around a bobbin (e.g., a sleeve 718). The magnetic field coil 708 is placed close to the electrode 704, the plasma chamber (region) 706, the outlet hole 711, and/or the plasma arc source signal. In some embodiments, the magnetic field coil 708 generally surrounds a substantial portion of the plasma chamber 706 adjacent the outlet hole 711. The magnetic field source signal received from the circuit driver board 703 causes the magnetic field coil 708 to control the direction of the plasma arc source signal to direct the plasma field from the plasma chamber 706 onto a target spot 722 on the target tissue 715.

In some embodiments, the inert gas chamber 709 contains a store of the (inert) gas or species in which the plasma is generated. The gas tube 710 physically connects the inert gas chamber 709 to one end of a passageway 712 through the electrode 704. (In some embodiments, the gas tube 710 extends through the passageway 712 through the electrode 704.) When the gas flow valve 717 is activated by the valve control signal, the inert gas passes from the inert gas chamber 709, through the gas tube 710, through the passageway 712, out an electrode outlet hole 713 at the other end of the passageway 712, and into the plasma chamber 706. In some embodiments, the gas contained in the gas chamber 709 is chosen to create particular reactive species within the plasma.

In some embodiments, the magnetic field coil 708 represents at least two separate plasma arc steering and focusing elements. In some embodiments, there is a steering coil and a focusing coil, each comprising a magnetic field coil.

Not to be limited by theory, the spot size will be a primary factor in determining the power density of the plasma arc stream where it interacts with the target tissue being treated. In some embodiments, the spot size can be used to generate a particular average temperature rise in the tissue that is interacting with the plasma arc stream within the target spot 722. With a smaller target spot size, the treatment subject is typically able to withstand a greater temperature and associated pain. In some embodiments, the target spot diameter has a predetermined size from 0.1 mm to 10 mm, from 0.5 to 5 mm, or from 1 mm to 2 mm, or 10, 5 or 2 mm or less.

In some embodiments, the device is scanned manually by the user (i.e., moving the target spot 722 manually to interact with all of the areas of the target tissue 715 intended to be treated). In some embodiments, the plasma arc stream rasters back and forth over the tissue to be treated, e.g., using the steering coil to control the direction of the plasma arc stream and scan the target spot 722 over an area larger than the target spot 722. Not to be limited by theory, the temperature of the tissue interacting with the plasma arc stream can be impacted or controlled by the amount of time that the plasma arc stream is interacting with the target tissue 715, up until an amount of time where a steady state temperature is reached. In some embodiments, the scanning rate of the plasma arc stream over the target tissue 715 being treated is chosen to generate a particular average temperature rise in the target tissue 715 that is interacting with the plasma arc stream.

In some embodiments, the insulators 705 are made of insulating plastic materials, ceramics, glass or other appropriate insulating materials. The insulators 705 contact the inside surface of the housing 701 and the outer surface of the sleeve 718 or the electrode 704 (near the two ends of the electrode 704) to support the electrode 704 (and the magnetic field coil 708) in a spaced apart, generally concentric, relationship with the housing 701. The insulators 705, thus, electrically and thermally insulate the electrode 704 (and the magnetic field coil 708) from the inside surface of the housing 701.

In some embodiments, the battery 702 is any appropriate rechargeable or non-rechargeable power storage source (e.g., a Li-ion battery, an alkaline battery, etc.) capable of producing an appropriate voltage (e.g., 0.8 volts dc to 7.4 volts dc). The battery 702 is electrically connected to the circuit driver board 703 to provide electrical power to the circuit driver board 703.

In some embodiments, the start/stop switch (or button) 707 is mounted through the housing 701, so that it is exposed and accessible on the outside of the housing 701. Inside the housing 701, the start/stop switch 707 is electrically connected to the circuit driver board 703. When turned on or off (e.g., by being pressed or switched by a user), the start/stop switch 707 activates or deactivates, respectively, the operation of the circuit driver board 703.

When activated, the circuit driver board 703 provides the plasma arc source signal to the electrode 704, the magnetic field source signal to the magnetic field coil 708, and the valve control signal to the gas flow valve 717. In response, the inert gas flows (as described above) to the plasma chamber 706, and the electrode 704 and the magnetic field coil 708 generate the plasma in the inert gas in the plasma chamber 706. As the gas flows through the plasma chamber 706, the generated plasma passes out through the outlet hole 711 as a plasma arc stream 714. When the outlet hole 711 is held near the target tissue 715 (e.g., a person's toenail) the plasma arc stream 714 heat treats the target tissue (e.g., to kill a fungus 716 on the target tissue 715). In some embodiments, the gas contained in the gas chamber 709 is chosen to tune the temperature of the plasma, which in turn affects the target temperature of the target tissue being treated while interacting with the plasma. In some embodiments, the target temperature and the temperature differential are the same or similar to that described above for embodiments of FIGS. 1-3 and/or 4-6. In some embodiments, the heat generated in the target tissue 715 causes, the cavitation and resulting effects, as described above.

In some embodiments, the plasma arc source signal provided by the circuit driver board 703 to the electrode 704 is continuous wave or pulsed. In some embodiments, the plasma arc source signal provided to the electrode 704 has a time-averaged power, a fundamental frequency, a pulsing frequency, a pulsing duty cycle, pulse duration, and/or a pulse shape that are the same or similar to the parameters described above for embodiments of FIGS. 1-3 and/or 4-6. Not to be limited by theory, the combination of the time-averaged power, the fundamental frequency, the pulsing frequency, the pulsing duty cycle, and the pulse shape generally determine the average temperature rise of the target tissue interacting with the plasma arc stream. In some embodiments, the time-averaged power, the fundamental frequency, the pulsing frequency, the pulsing duty cycle, and the pulse shape of the plasma arc source signal provided to the electrode 704 are chosen or tuned to generate a particular predetermined average temperature rise in the target tissue that is interacting with the resulting plasma arc stream. In this manner, the circuit driver board 703 controls the temperature or temperature increase of the target tissue by controlling, adjusting or modulating these parameters. In some embodiments, the target temperature can be different, as described above. In some embodiments, different plasma parameters (e.g., time-average power, pulsing frequency, etc.) are needed to generate a particular predetermined target temperature in a target tissue interacting with the plasma arc stream.

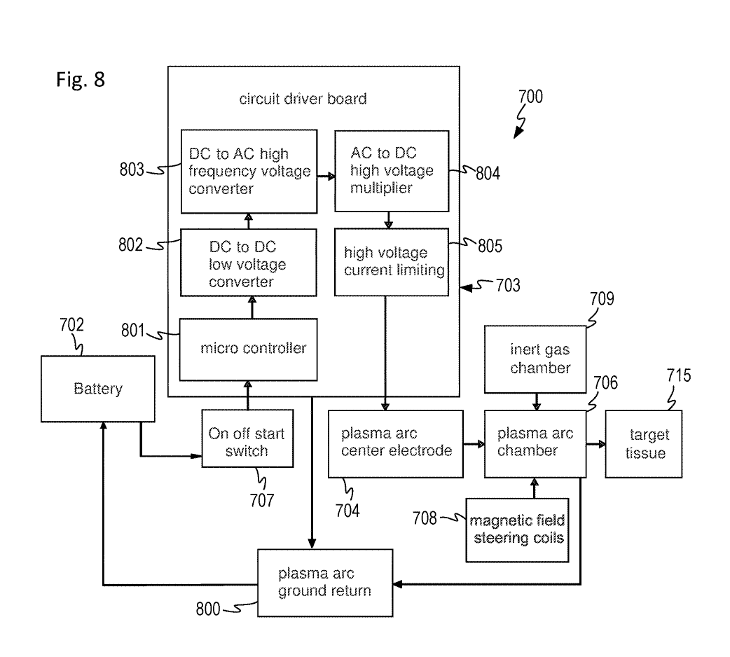

FIG. 8 shows an embodiment of the treatment device 700 having the battery 702, the circuit driver board 703, the one or more electrode 704, the plasma (arc) chamber 706, the start/stop switch 707, the magnetic field coil 708, and the inert gas chamber 709. In some embodiments, these components are generally the same or similar to the components (having the same reference numbers) described above with reference to FIG. 7. Additionally, in some embodiments, the treatment device 700 also has a plasma arc ground return 800. Furthermore, in some embodiments, the circuit driver board 703 generally has a microcontroller 801, a DC to DC low voltage converter 802, a DC to AC high frequency voltage converter 803, an AC to DC high voltage multiplier 804, and a high voltage current limiting element 805.

In some embodiments, the plasma arc ground return 800 is a wire or other conductor that extends between and electrically connects the plasma arc chamber 706, the circuit driver board 703, and the battery 702 to complete the electrical circuit. In some embodiments, the housing 701 serves as the plasma arc ground return 800. In some embodiments, another ground return (not shown in FIG. 8) is provided for the grounding pad 719 attached to the treatment subject, as described above, to assist in ensuring that the plasma arc stream reaches the target tissue.

In some embodiments, the microcontroller 801 controls all functions of operation of the treatment device 700, including the magnetic field coil 708 and the direction of the plasma arc signal. Under this control, the microcontroller 801 can narrow the plasma field to a pinpoint or open it to a wider field.

In some embodiments, the DC to DC low voltage converter 802 receives a voltage from the battery 702 and converts it to a DC voltage(s) that is utilized by the components of the circuit driver board 703.

In some embodiments, the DC to AC high frequency voltage converter 803 is a relatively efficient means in a relatively small package size for receiving the DC voltage from the DC to DC low voltage converter 802 and generating an AC high frequency voltage signal that is needed for plasma arc signal generation.

In some embodiments, the AC to DC high voltage multiplier 804 receives the AC high frequency voltage signal and converts it to further increase the voltage and rectify it to a DC voltage.

In some embodiments, the high voltage current limiting element 805 receives the DC voltage and limits the power of the plasma arc, so as to protect the target tissue 715 from temperatures greater than the target temperature.

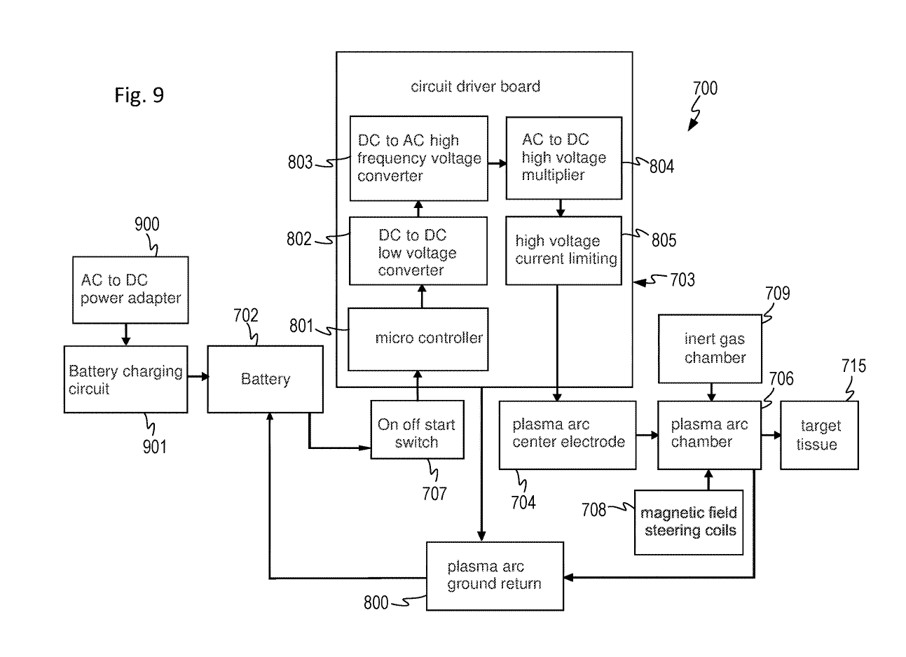

FIG. 9 shows an embodiment of the treatment device 700 having the battery 702, the circuit driver board 703, the one or more electrode 704, the plasma (arc) chamber 706, the start/stop switch 707, the magnetic field coil 708, the inert gas chamber 709, the plasma arc ground return 800, the microcontroller 801, the DC to DC low voltage converter 802, the DC to AC high frequency voltage converter 803, the AC to DC high voltage multiplier 804, and the high voltage current limiting element 805. In some embodiments, these components are generally the same or similar to the components (having the same reference numbers) described above with reference to FIGS. 7 and 8. In some embodiments, the treatment device 700 also has or is connected to an AC to DC power adapter 900 and a battery charging circuit 901.

In some embodiments, the AC to DC power adapter 900 is used for charging the battery 702 and for powering the treatment device 700 when plugged into a AC outlet in the event the battery 702 was totally or partially discharged. In some embodiments, the AC to DC power adapter 900 is external or internal to the housing 701.

In some embodiments, the battery charging circuit 901 is incorporated onto the circuit driver board 703 for quick charging of the battery 702. In some embodiments, the battery charging circuit 901 is external or internal to the housing 701.

Although embodiments of the present invention have been discussed primarily with respect to specific embodiments thereof, other variations are possible. Various configurations of the described system may be used in place of, or in addition to, the configurations presented herein. For example, additional components may be included in circuits where appropriate. As another example, configurations were described with general reference to certain types and combinations of circuit or system components, but other types and/or combinations of circuit components could be used in addition to or in the place of those described.

Those skilled in the art will appreciate that the foregoing description is by way of example only, and is not intended to limit the present invention. Nothing in the disclosure should indicate that the present invention is limited to systems that have the specific type of devices shown and described. Nothing in the disclosure should indicate that the present invention is limited to systems that require a particular form of integrated circuits or hardware components, except where specified. In general, any diagrams presented are only intended to indicate one possible configuration, and many variations are possible. Those skilled in the art will also appreciate that methods and systems consistent with the present invention are suitable for use in a wide range of applications.

While the specification has been described in detail with respect to specific embodiments of the present invention, it will be appreciated that those skilled in the art, upon attaining an understanding of the foregoing, may readily conceive of alterations to, variations of, and equivalents to these embodiments. These and other modifications and variations to the present invention may be practiced by those skilled in the art, without departing from the scope of the present invention.

What is claimed is:

1. A tissue treatment device comprising:
a housing that includes an outlet hole;
a circuit driver disposed within the housing and that generates a plasma arc source signal;
an electrode disposed within the housing and that receives the plasma arc source signal and generates a plasma stream in response to the plasma arc source signal; and
a plasma arc focusing element disposed within the housing adjacent the electrode and the outlet hole and that focuses the plasma stream to pass through the outlet hole and onto a target spot on a target tissue of a treatment subject for heat treatment of the target tissue.

2. The tissue treatment device of claim 1, further comprising:
a plasma arc steering element disposed within the housing adjacent the electrode and the outlet hole and that controls a direction of the plasma stream to scan the target spot over an area larger than the target spot.

3. The tissue treatment device of claim 2, wherein:
the plasma arc focusing element and the plasma arc steering element comprise a single magnetic field coil.

4. The tissue treatment device of claim 2, wherein:
the plasma arc focusing element comprises a first magnetic field coil; and
the plasma arc steering element comprises a second magnetic field coil.

5. The tissue treatment device of claim 1, wherein:
the plasma arc focusing element narrows the plasma stream to a diameter of 2 mm or less for the target spot.

6. The tissue treatment device of claim 1, wherein:
the circuit driver controls a temperature increase of the target tissue that is caused by the plasma stream by modulating a fundamental frequency and power level of the plasma arc source signal.

7. The tissue treatment device of claim 6, wherein:
the circuit driver further controls the temperature increase by modulating a pulsing frequency and pulsing duty cycle at which the plasma arc source signal is pulsed.

8. The tissue treatment device of claim 1, wherein:
the plasma stream is capable of generating a cavitation pressure wave under a surface of the target tissue at the target spot.

9. The tissue treatment device of claim 1, wherein:
the heat treatment is capable of treating a medical condition of the target tissue that is at least one of: a fungal infection, a bacterial infection, a pimple, and a fever blister.

10. The tissue treatment device of claim 1, further comprising:
a grounding pad electrically connected to the circuit driver through the housing, wherein the grounding pad is capable of contacting a skin surface of the treatment subject to form an electrical return path.

11. The tissue treatment device of claim 1, further comprising:
a gas chamber disposed within the housing and that contains an inert gas that is flowed to the outlet hole, wherein the plasma stream is generated in the inert gas.

12. The tissue treatment device of claim 1, further comprising:
a power storage source disposed within the housing and electrically connected to the circuit driver to provide electrical power to the circuit driver to generate the plasma arc source signal.

13. A tissue treatment device comprising:
a housing that includes an outlet hole;
a circuit driver disposed within the housing and that generates a plasma arc source signal; and
an electrode disposed within the housing and that receives the plasma arc source signal and generates a plasma stream in response to the plasma arc source signal;
wherein:
the plasma stream passes through the outlet hole and onto a target spot on a target tissue of a treatment subject for heat treatment of the target tissue; and
the circuit driver controls a temperature increase of the target tissue that is caused by the plasma stream by modulating a power level of the plasma arc source signal.

14. The tissue treatment device of claim 13, wherein:
the circuit driver further controls the temperature increase by modulating a fundamental frequency of the plasma arc source signal.

15. The tissue treatment device of claim 14, wherein:
the circuit driver further controls the temperature increase by modulating a pulsing frequency or pulsing duty cycle at which the plasma arc source signal is pulsed.

16. The tissue treatment device of claim 13, further comprising:
a plasma arc focusing element disposed within the housing adjacent the electrode and the outlet hole and that focuses the plasma stream to pass through the outlet hole and onto the target spot.

17. A method comprising:
generating, by a circuit driver disposed within a housing of a tissue treatment device, a plasma arc source signal;
generating, by an electrode disposed within the housing, a plasma stream in response to the plasma arc source signal;
generating, by a plasma arc focusing element disposed within the housing, a magnetic field source signal;
focusing and directing, by the magnetic field source signal, the plasma stream through an outlet hole of the housing and onto a target spot of a predetermined size on a target tissue of a treatment subject for heat treatment of the target tissue.

18. The method of claim 17, further comprising:
altering the directing of the plasma stream to scan the target spot over an area larger than the target spot.

19. The method of claim 17, further comprising:
controlling a temperature increase of the target tissue that is caused by the plasma stream by modulating, by the circuit driver, a fundamental frequency and power level of the plasma arc source signal.

20. The method of claim 19, further comprising:
controlling the temperature increase by also modulating a pulsing frequency and pulsing duty cycle at which the plasma arc source signal is pulsed.
